(19) United States
(12) Patent Application Publication
(10) Pub. No.: US 2008/0147168 A1
(43) Pub. Date: Jun. 19, 2008

(54) Intravascular implantable device having detachable tether arrangement

(76) Inventors: Terrance Ransbury, Chapel Hill, NC (US); Kevin Holbrook, Chapel Hill, NC (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/999,517

(22) Filed: Dec. 4, 2007

Related U.S. Application Data
(60) Provisional application No. 60/868,434, filed on Dec. 4, 2006.
(60) Provisional application No. 60/868,437, filed on Dec. 4, 2006.

(51) Int. Cl. A61F 2/06

(52) U.S. Cl. 623/1.15; 623/1.1; 623/1.36

(57) ABSTRACT

An intravascular implantable medical device includes a detachable tether arrangement. In one embodiment, the detachable tether arrangement comprises a tip assembly that includes a break-away joint and a tether portion, and may also include a connector and an extension portion connected to an end of an elongated intravascular implantable medical device. In this embodiment, the break-away joint is disposed between the extension portion and the tether portion. In some embodiments, the extension portion may comprise a housing or other arrangement that includes an antenna for transmitting and receiving data. In other embodiments, the extension portion may also include a lead for defibrillation, pacing, and/or sensing cardiac electrical activity. In certain embodiments, the tether arrangement is designed to be secured within a patient's vasculature with a vascular anchor either in the form of a separate anchor, such as a conventional stent, or by various integrated retention arrangements.

### RELATED APPLICATIONS

The present application claims the benefit of U.S. Provisional Application No. 60/868,434, filed Dec. 4, 2006, and U.S. Provisional Application No. 60/868,437, filed Dec. 4, 2006, and U.S. Provisional Application titled “Implantation Methods, Systems and Tools for Intravascular Implantable Devices”, filed Dec. 3, 2007, the disclosures of which are hereby incorporated by reference in their entireties.

### FIELD OF THE INVENTION

The present invention relates generally to surgical devices and methods for retaining medical devices within the body, and more specifically to a detachable tether arrangement for a medical device and a method of extracting an intravascular implantable medical device from within a vessel.

### BACKGROUND OF THE INVENTION

Implantable medical devices such as pacemakers, defibrillators, and implantable cardioverter defibrillators (“ICDs”) have been successfully implanted in patients for years for treatment of heart rhythm conditions. Pacemakers are implanted to detect periods of bradycardia and deliver low energy electrical stimuli to increase the heart rate. ICDs are implanted in patients to cardiovert or defibrillate the heart by delivering high energy electrical stimuli to slow or reset the heart rate in the event a ventricular tachycardia (VT) or ventricular fibrillation (VF) is detected. Another type of implantable device detects an atrial fibrillation (AF) episode and delivers electrical stimuli to the atria to restore electrical coordination between the upper and lower chambers of the heart. Still another type of implantable device stores and delivers drug ad/or gene therapies to treat a variety of conditions, including cardiac arrhythmias. The current generation for all of these implantable devices are typically can-shaped devices implanted under the skin that deliver therapy via leads that are implanted in the heart via the patient's vascular system.

Next generation implantable medical devices may take the form of elongated intravascular devices that are implanted within the patient's vascular system, instead of under the skin. Examples of these intravascular implantable devices are described, for example, in U.S. Pat. No. 7,082,336, U.S. Published Patent Application Nos. 2005/0043765A1, 2005/0208471A1 and 2006/0217779A1. These devices contain electric circuitry and/or electronic components that are hermetically sealed to prevent damage to the electronic components and the release of contaminants into the bloodstream. Due to the length of these implantable devices, which in some cases can be approximately 10-60 cm in length, the devices generally are designed to be flexible enough to move through the vasculature while being sufficiently rigid to protect the internal components.

The issue of how to secure such an implantable device in the vasculature is one of the challenges for this next generation of intravascular implantable devices. In addition to the mechanical and operational considerations related to an anchoring system, there are physical and biological implications for the patient, as well as considerations for how an anchoring system may affect the manner in which the implantable device delivers therapy.

As described in some of the embodiments shown in U.S. Pat. No. 7,082,336 and U.S. Published Patent Application No. 2004/0249431, the anchoring system was arranged proximate the middle of the intravascular implantable device so as to be positioned in the vena cava within the thorax. This arrangement anchored the intravascular implantable device near the middle of the patient's torso at a location generally corresponding to the diaphragm. In some embodiments, the anchoring system was integrated with the body of the intravascular implantable device. In other embodiments, the anchoring system was a separate device, such as a stent, that was used to pin the body of the intravascular implantable device in position between the stent and the vessel wall. In still other embodiments, a lead extending from a distal end of the body of the intravascular device would also be anchored in the vasculature, such as in a subclavian vein.

An alternative integrated anchoring system for an intravascular implantable device is described in some of the embodiments shown in U.S. Published Patent Application No. 2005/0208471A1. This alternative integrated anchoring system utilized a radially expandable member positioned proximate the middle of the body of the device to secure the device. In some embodiments, the radially expandable member centered the device within the diameter of the vessel. In other embodiments, two or more radially expandable members were used to secure the middle of the body of the device within a vessel.

The approaches of securing an intravascular implantable device within the thorax by an anchoring system proximate the middle of the body of the device and positioned in the vena cava generally corresponding to the diaphragm of the patient were intended to create a secure and balanced anchoring of the device within the largest diameter vessel in the body. These approaches sought to reduce issues of thrombosis and potential dislodgement of the anchoring system due to impact or movement of the patient.

In many applications, intravascular implantable devices are intended to provide temporary or initial treatment for a condition, and the devices are not intended to remain implanted for the remainder of the patient's life. Therefore, it may become necessary to extract or explant the intravascular implantable device during the life of a patient. With the existing approaches for securing an intravascular device, the device commonly becomes fused to the anchor and/or to the vasculature due to thrombus formation and/or endothelialization,

Current devices and methods of removal of intravascular implantable devices can make explantation of the device a difficult process. Most of the techniques and apparatus developed for explantation of implanted medical devices have focused on removal of devices, such as pacemaker or defibrillator leads, that are implanted within an organ like the heart. As a result, very little effort has been devoted to the design of intravascular implantable devices that would facilitate their explantation. It would be desirable to provide for an improved intravascular device that is easily removed, and to provide for an improved method of explantation of an intravascular implantable device.

### SUMMARY OF THE INVENTION

The present invention is directed to an intravascular implantable medical device that includes a detachable, severable, releasable, or otherwise removable tether arrangement. In one embodiment, the detachable tether arrangement comprises a tip assembly that includes a break-away joint and a tether portion, and may also include a connector and an extension portion connected to an end of an elongated intravascular implantable medical device. In this embodiment, the break-away joint is disposed between the extension portion and the tether portion. The break-away joint may comprise a snap-fit, threaded joint, or other similar configurations. In some embodiments, the extension portion may comprise a housing or other arrangement that includes an antenna for transmitting and receiving data. In other embodiments, the extension portion may also include a lead for defibrillation, pacing, and/or sensing cardiac electrical activity. In certain embodiments, the tether arrangement is designed to be secured within a patient's vasculature with a vascular anchor either in the form of a separate anchor, such as a conventional stent, or by various integrated retention arrangements.

In one embodiment, the vascular anchor is separate from the implantable device and captures a tether portion that extends from the implantable device between the anchor and the vasculature. In another embodiment, the anchor may be incorporated as part of the implantable device. In one embodiment, the vascular anchor and/or the implantable device include mechanisms to optimize interference between the anchor and the tether portion in a way that does not induce a rupture of the vessel while providing for adequate clinical attachment of the implantable device within the patient.

Unlike the previous approaches to extracting an intravascular implantable device anchored near the middle of the patient's torso, embodiments of the present invention utilize a detachable tether arrangement positioned proximate and end of the elongated body portion of the intravascular implantable device which does not require that the anchoring arrangement be extracted in order to explant the body portion of the intravascular implantable device. In one embodiment, the detachable tether arrangement is utilized in conjunction with a superior anchoring arrangement that enables primary explantation access via a femoral puncture to retrieve the intravascular implantable device by detaching the tether portion at the break-away joint. A secondary, fallback access that can occur by a subclavian vessel puncture in the event that the primary explantation access is unsuccessful in retrieving the intravascular implantable device. The secondary access affords a more direct access to the detach/release mechanisms of the tether arrangement and the anchor location, and also permits control of both ends of the intravascular implantable device during the explantation procedure.

In accordance with the present invention, the intravascular implantable device may be extracted by disconnecting, detaching or otherwise releasing the break-away joint and removing the body portion of the intravascular implantable device. In one embodiment, the tether portion and the anchor may remain secured within the vasculature and need not be explanted. In one embodiment, the tip assembly includes a break-away joint non-releasably coupled to the tether portion, and adapted to be releasably coupled to an intravascular implantable medical device. The intravascular implantable device may be any one or a combination of defibrillator, cardioverter, pacemaker, monitor or drug/gene therapy delivery device and may be either a temporary or permanent device.

In one embodiment, the present invention comprises a method of extracting an implantable intravascular device. The method comprises providing an intravascular implantable device having a detachable tip assembly on one end, the tip assembly including a break-away joint releasably coupled to the device, and a tether fixedly secured to the break-away joint, the tether being secured within the vasculature by an anchor. Detaching the device from the joint allows the device to be removed, such as through an incision in a femoral vein, while the tether and joint remain anchored within the vasculature. In a further embodiment, the implantable intravascular device includes an extension portion between the device body and the break-away joint such that when the device is extracted, the extension portion is extracted with the device.

The above summary of the various embodiments of the invention is not intended to describe each illustrated embodiment or every implementation of the invention. This summary represents a simplified overview of certain aspects of the invention to facilitate a basic understanding of the invention and is not intended to identify key or critical elements of the invention or delineate the scope of the invention.

### BRIEF DESCRIPTION OF THE DRAWINGS

The invention may be more completely understood in consideration of the following detailed description of various embodiments of the invention in connection with the accompanying drawings, in which:

FIG. 1 is a perspective illustration depicting human cardiac anatomy.

FIG. 2 is a cross-sectional plan view of an implantable intravascular pacing device according to an example embodiment of the present invention.

FIG. 2A is a schematic representation of FIG. 2.

FIG. 3 is a cross-sectional plan view of an implantable intravascular pacing device according to an example embodiment of the present invention.

FIG. 3A is a schematic representation of FIG. 3.

FIG. 4 is a cross-sectional plan view of an implantable intravascular pacing device according to an example embodiment of the present invention.

FIG. 5 is a cross-sectional plan view of an implantable intravascular pacing device according to an example embodiment of the present invention.

FIG. 5A is a schematic representation of FIG. 5.

FIG. 6 is a cross-sectional plan view of an implantable intravascular defibrillation device according to an example embodiment of the present invention.

FIG. 6A is a schematic representation of FIG. 6.

FIG. 7 is a perspective view an implantable intravascular defibrillation device according to an example embodiment of the present invention.

FIG. 8 is a plan view of a detachable tip assembly according to an example embodiment of the present invention.

FIG. 9 is a cross-sectional view taken along the line A-A in FIG. 8.

FIG. 10 is a close-up view of detail area B in FIG. 9.

FIG. 11 is an exploded view of an example embodiment of a detachable tip assembly according to an example embodiment of the present invention.

FIG. 12 is an exploded view of detachable tip assembly components according to an example embodiment of the present invention.

FIG. 13 is a plan view of a detachable barb tip portion according to an example embodiment of the present invention.

FIG. 14 is a plan view of a component of a detachable tip assembly according to an example embodiment of the present invention.

FIG. 14A is a plan view of a component of a detachable tip assembly according to an example embodiment of the present invention.

FIG. 14B is a plan view of a component of a detachable tip assembly according to an example embodiment of the present invention.

FIG. 15 is a perspective view of a component of a detachable tip assembly according to an example embodiment of the present invention.

FIG. 15A is a side view of the component in FIG. 15, in combination with an anchor.

FIG. 16 is a cross-sectional view of a detachable tip assembly according to an example embodiment of the present invention.

FIG. 17 is a side perspective view of an implanted medical device having a detachable tip assembly according to an example embodiment of the present invention.

FIG. 18 is a close-up perspective view of an implanted medical device having a detachable tip assembly according to an example embodiment of the present invention.

FIG. 19 is a cross-sectional view taken along the line 19-19 in FIG. 18.

FIG. 20 is a perspective view of an implanted medical device having a detachable tip assembly according to an example embodiment of the present invention.

FIG. 21 is a perspective view of an implanted medical device having a detachable tip assembly according to an example embodiment of the present invention.

FIG. 22 is a perspective view of a detachable tip assembly according to one embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the claims.

### DETAILED DESCRIPTION OF THE EMBODIMENTS

In the following detailed description of the present invention, numerous specific details are set forth in order to provide a thorough understanding of the present invention. However, one skilled in the art will recognize that the present invention may be practiced without these specific details. In other instances, well-known methods, procedures, and components have not been described in detail so as to not unnecessarily obscure aspects of the present invention.

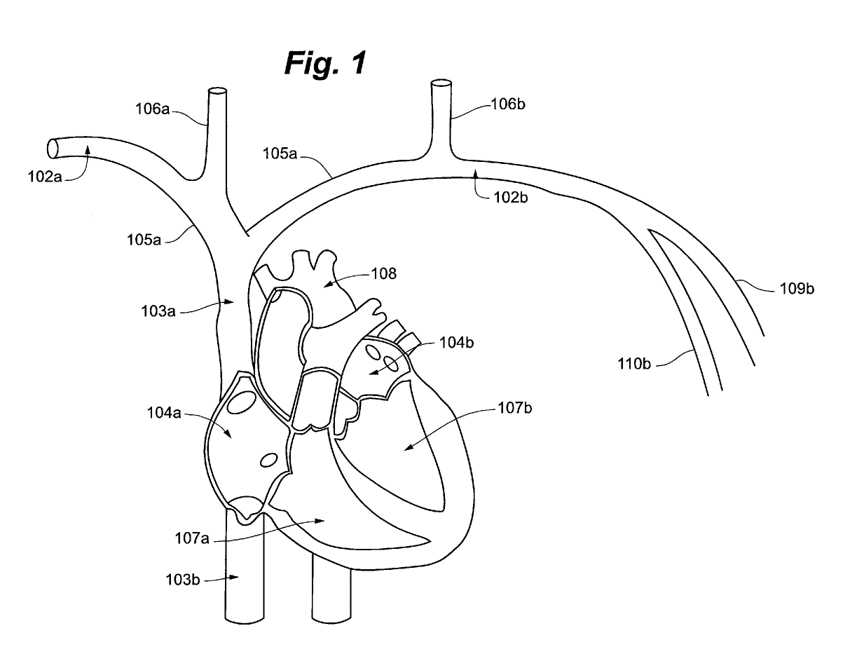

Referring now to FIG. 1, the general cardiac anatomy of a human is depicted, including the heart and major vessels. The following anatomic locations are shown and identified by the listed reference numerals: Right Subclavian 102a, Left Subclavian 102b, Superior Vena Cava (SVC) 103a, Inferior Vena Cava (IVC) 103b, Right Atrium (RA) 104a, Left Atrium (LA) 104b, Right Innominate/Brachiocephalic Vein 105a, Left Innominate/Brachiocephalic Vein 105b, Right Internal Jugular Vein 106a, Left Internal Jugular Vein 106b, Right Ventricle (RV) 107a, Left Ventricle (LV) 107b, Aortic Arch 108, Descending Aorta 109, Right Cephalic Vein 109a (not shown in FIG. 1), Left Cephalic Vein 109b, Right Axillary Vein 110a (not shown in FIG. 1) and Left Axillary Vein 110b.

One embodiment of the present invention describes intravascular electrophysiological systems that may be used for a variety of functions to treat cardiac arrhythmias with electrical stimulation. These functions include defibrillation, pacing, and/or cardioversion. In general, the elements of an intravascular implantable device for electrophysiological therapy include at least one device body and typically, but optionally, at least one lead coupled to the body. While the present invention is directed to anchoring and retention of the device body of an intravascular implantable device, it will be understood that, in some embodiments, the one or more leads may also be anchored or retained in the vasculature or within the heart. Alternatively, the intravascular implantable device may have no leads, such as for an embodiment of an intravascular implantable drug/gene therapy device, or the one or more leads may not be anchored or retained in the vasculature or within the heart.

Various examples of intravascular implantable electrophysiology devices, such as intravascular defibrillation and/or pacing devices 20 and leads 28 will be given in this description. In those examples, reference numerals such as 20a, 20b, 20c, etc., will be used to describe certain embodiments of the intravascular device 20, whereas elsewhere reference numeral 20 may be used to more generally refer to intravascular devices of the type that may be used with the present invention for providing therapy other than, or in addition to, cardiac electrophysiology. Likewise, reference number 28 may be used generally to refer to leads of a type that may be used with the system. Reference number 100 refers generally to vessels and/or vessel walls within the human body.

In one embodiment, device 20 includes components, known in the art to be necessary to carry out the system functions of an implantable electrophysiology device. For example, device 20 may include one or more pulse generators, including associated batteries, capacitors, microprocessors, communication circuitry and circuitry for generating electrophysiological pulses for defibrillation, cardioversion and/or pacing. Device 20 may also include detection circuitry for detecting arrhythmias or other abnormal activity of the heart. The specific components to be provided in device 20 will depend upon the application for the device, and specifically whether device 20 is intended to perform defibrillation, cardioversion, and/or pacing along with sensing functions.

Device 20 can be proportioned to be passed into the vasculature and to be anchored within the vasculature of the patient with minimal obstruction to blood flow. Suitable sites for introduction of device 20 into the body can include, but are not limited to, the venous system using access through the right or left femoral vein or the right or left subclavian vein. For purposes of describing the present invention, the various portions of the device 20 will be referenced to the location of those portions, the proximal portion 22, the distal portion 24 and the middle portion 26 relative to the introduction site in the femoral vein. Device 20 generally includes a proximal end and a distal end. It will be understood, however, that if an alternate access site were used to introduce the device 20, such as the subclavian veins, the various portions 22, 24 and 26 of the device 20 would be referenced relative to the inferior/superior location of the device 20 within the vascular system in the torso of a patient. In one embodiment, distal portion 24 may be defined as being part of the device body, encompassing up to the distal-most third of the body of device 20. In another embodiment, distal portion 24 may be defined as encompassing part of the body of device 20 and part of tether 52. In a further embodiment, distal portion 24 is defined as not encompassing the device body at all, rather it encompasses tether 52.

In one embodiment, the device 20 can have a streamlined maximum cross sectional diameter which can be in the range of 3-15 mm or less, with a maximum cross-sectional diameter of 3-8 mm or less in one embodiment. The cross-sectional area of device 20 in the transverse direction (i.e. transecting the longitudinal axis) can preferably be as small as possible while still accommodating the required components. This area can be in the range of approximately 79 mm² or less, in the range of approximately 40 mm² or less, or between 12.5-40 mm², depending upon the embodiment and/or application.

In one embodiment, the cross-section of device 20 (i.e., transecting the longitudinal axis) may have a circular cross-section, although other cross-sections including crescent, flattened, or elliptical cross-sections may also be used. It can be highly desirable to provide the device with a smooth continuous contour so as to avoid voids or recesses that could encourage thrombus formation on the device. It can also be desirable to provide for a circular cross-section to aid in removal or explantation of the device that more easily permits the device to be torqued or rotated during the removal or explantation to break free of any thrombosis or clotting that may have occurred. In one embodiment, the cross-section of device 20 is generally isodiametric along the entirety of its longitudinal length other than for tapered portions at the proximal and distal ends of the device 20. In one embodiment, the aspect ratio of the cross-sectional diameter to the longitudinal length of each container is less than at least 1.5:2 (e.g., 15 mm diameter to 20 mm length) and in another embodiment the aspect ratio is at least 1:4.

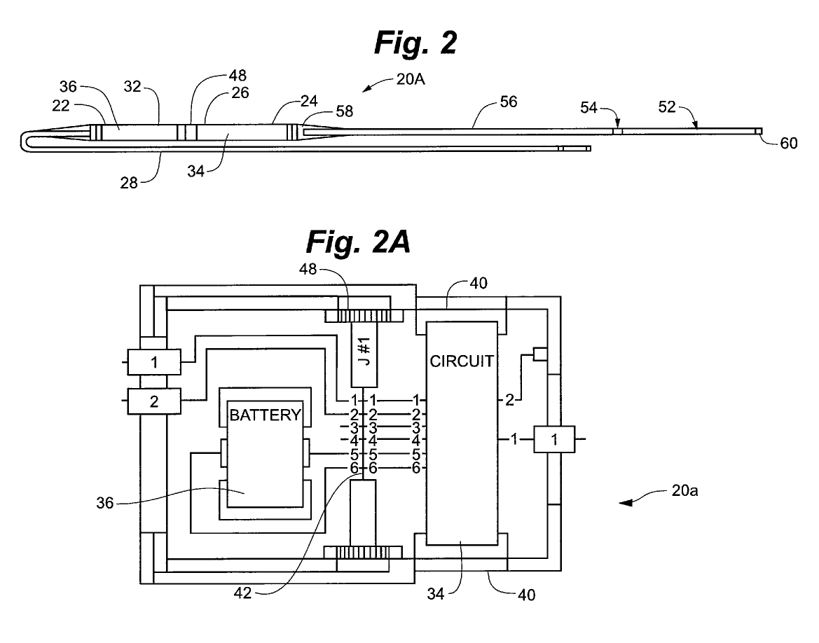

In one embodiment, the housing of device 20 may be covered by an electrically insulative layer or coating such as ePTFE. It may be desirable to provide a coating that is anti-thrombogenic (e.g., perfluorocarbon coatings applied using supercritical carbon dioxide) so as to prevent thrombus formation on device 20. It may also be beneficial that the coating have anti-proliferative properties so as to minimize endothelialization or cellular in growth, since minimizing growth into or onto device 20 will help minimize vascular trauma when the device is explanted. The coating may thus also be one which elutes anti-thrombogenic compositions (e.g., heparin sulfate) and/or compositions that inhibit cellular in growth and/or immunosuppressive agents. If the housing of device 20 is conductive, this layer or coating may be selectively applied or removed to leave an exposed electrode region on the surface of the housing where necessary, such as depicted in FIGS. 2-6A.

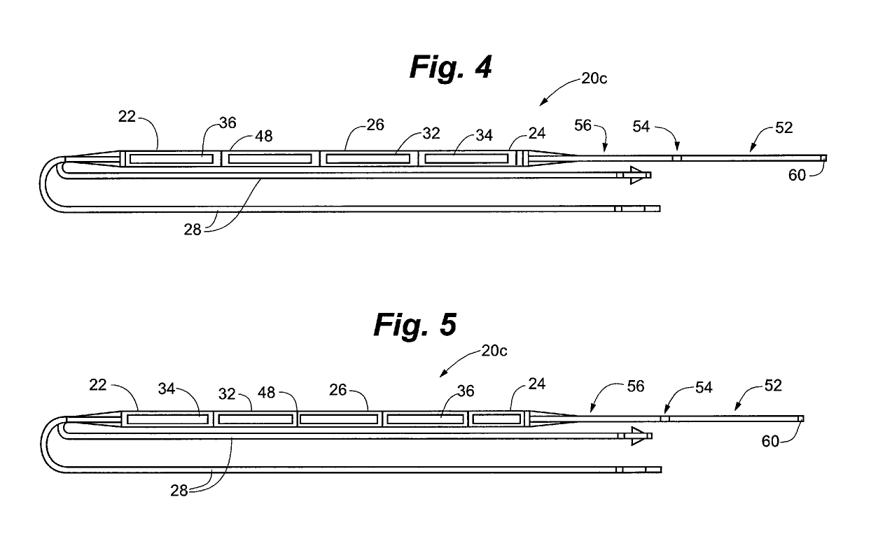

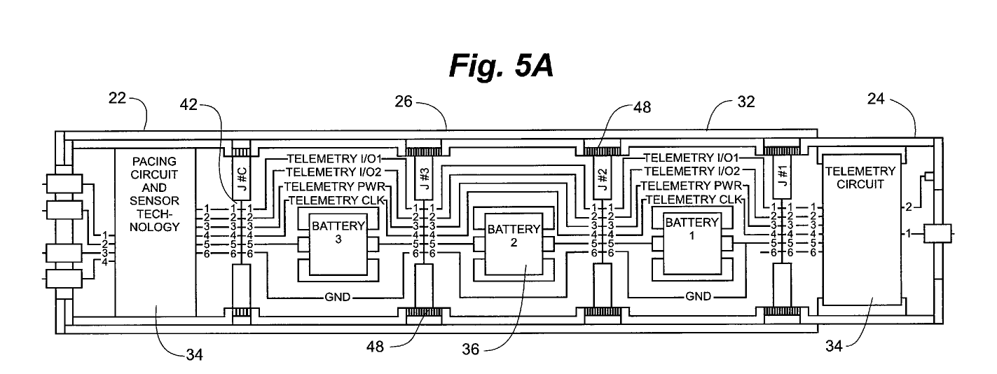

In some embodiments one or more leads 28 may extend from device 20 proximate any of the various portions 22, 24 and 26 of the device 20. In the embodiment shown in FIGS. 2, 3, and 6, for example, a single lead 28 is shown, extending from the proximal end 22 of device 20. A lead 28 includes one or more electrodes, such as tip electrodes, ring electrodes, or defibrillation electrodes. In embodiments having a tether 52, a lead 28 may be included within tether 52. If two leads 28 are used, they may extend from opposite ends of device 20, or they may extend from the same end of the device 20, such as depicted in FIGS. 4-5. Either or both of the leads may be equipped to sense electrical activity of the heart. Monitoring of the heart's electrical activity is needed to detect the onset of an arrhythmia. Activity sensed by the sensing electrode(s) is used by device 20 electronics to trigger delivery of a defibrillation shock that in one embodiment may be delivered via lead 28 having a defibrillation electrode or delivery of a pacing impulse that in one embodiment may be delivered via lead 28 via a pacing electrode.

The lead 28 may be a conventional defibrillation/pacing lead, although alternative lead configurations may be desirable if warranted by the desired placement of the device 20 and lead within the body. An optimal lead will preferably give the physician implanting the device flexibility to position the device at an appropriate location in the chosen vessel without concern that the leads extending from the device will not reach their intended location. Thus, for some patients it may be necessary to use a lead that is slightly longer than conventional leads, or the lead may include a coiled section that is similar to the configuration of a coiled telephone cord. A coiled section can allow elongation of the effective length of the lead when tension is applied to the coil. The coiled section or any alternate type of yieldable lead section may be a plastically deformable metal or polymer that will retain its extended configuration after it has been stretched to that configuration. Other configurations that will allow additional lead length to pull out from the device if needed may also be used.

For leads 28 that are to be positioned within a chamber of the heart, the lead may include a helical screw-in tip or be of the tined variety for fixation to the cardiac tissue. A detachable screw-in lead tip may be provided, which allows the lead tip to be left within the chamber of the heart when lead 28 is extracted.

Lead 28 may have a steroid-eluding tip to facilitate tissue in-growth for fixation purposes, or may include non-thrombogenic and/or non-proliferative surfaces or coatings similar to those as may be applied to device 20. For example, lead 28 may include a coating that is anti-thrombogenic (e.g. perfluorocarbon coatings applied using supercritical carbon dioxide) so as to prevent thrombus formation on the lead. It is also beneficial for the coating to have anti-proliferative properties so as to minimize endothelialization or cellular ingrowth, since minimizing growth into or onto the lead will help minimize vascular trauma when the device is explanted. The coating may thus also be one which elutes anti-thrombogenic compositions (e.g. heparin sulfate) and/or compositions that inhibit cellular in-growth and/or immunosuppressive agents.

It should be appreciated that in this disclosure the term “lead” is used to mean an element that includes conductors and electrodes in an elongated, sealed and insulated protective configuration that is adapted to withstand chronic implantation and is generally floppy in flexibility to permit the electrodes to be positioned somewhat remotely from the circuitry that energizes the electrodes via the conductors. The lead 28 may be integrated with the device body, or attachable to the device body in situ or prior to implantation, or the lead 28 may be integral with the device body as an extension of the device itself. Thus, leads may include elements that are simply extensions or tapers of the device 12a itself (such as the portion of the device 12a at which electrodes 22a are located) as well as more conventional leads. More than one lead 28 may be provided, and leads may be included on the proximal/inferior end of the device body, on the distal/superior portion of the device body, generally on the device body, and/or any combination thereof. In one embodiment, an end of the device body may be modified to include a stepped portion proximate the lead connection, such as on the proximal end of the device. The stepped portion allows a smooth transition between the exterior surface of the lead and the device body.

Given the minimal space allowed for components, it is desirable to arrange the components within device 20 so as to make efficient use of the available space. Examples of devices having space efficient arrangements of their contents are shown in FIGS. 2-6A. One example is identified by reference numeral 20a in FIG. 2. One embodiment of device 20a includes one or more elongate housings or enclosures 32 depicted in cross-section in FIG. 2A to allow the components housed within it to be seen. In one embodiment, enclosure 32 is a rigid or semi-rigid housing preferably formed of a material that is conductive, biocompatible, capable of sterilization and capable of hermetically sealing the components contained within the enclosure 32. One example of such a material is titanium, although other materials may also be used.

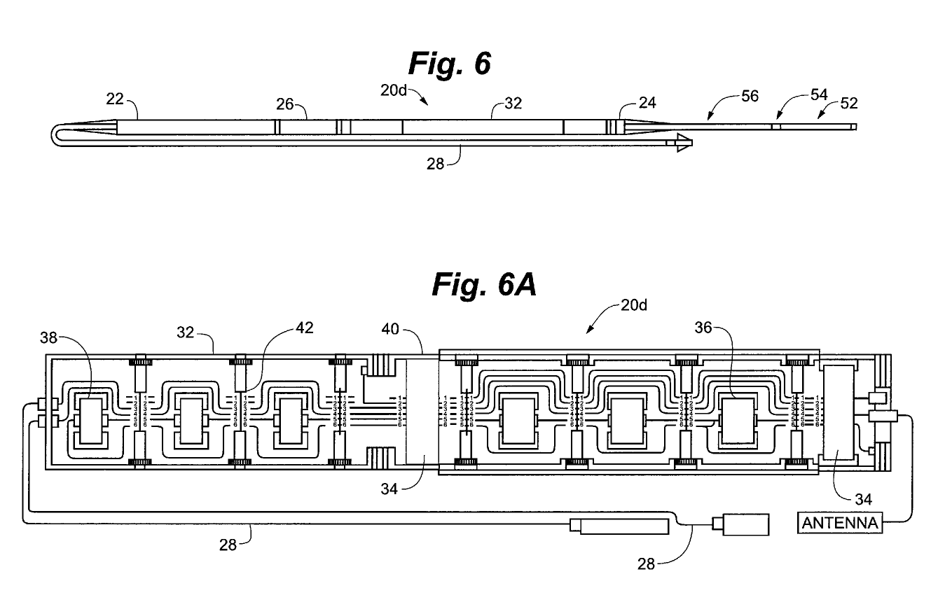

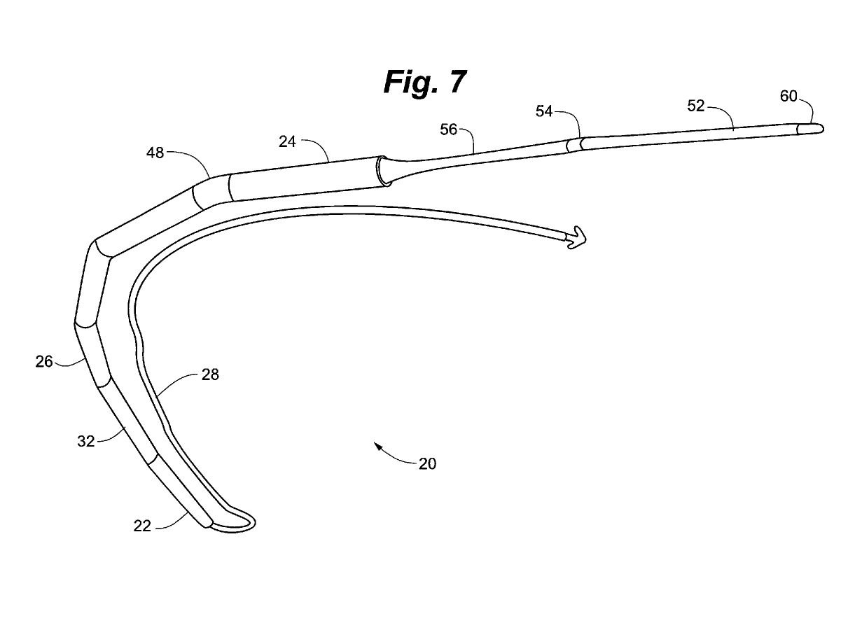

Within enclosure 32 are the electronic components 34 that govern operation of the device 20a. For example, components 34a are associated with delivery of a defibrillation pulse via a lead 28 (FIG. 6), whereas components 34b are associated with the sensing function performed using sensing electrodes on the defibrillation lead, on a separate lead 28 (e.g., FIGS. 4 and 5), or on the device body itself. Isolating components 34a from components 34b may be desirable if noise generated by the high voltage defibrillation circuitry 34a during charging might interfere with performance of the sensing circuitry 34b, or if practical limitations exist with respect to circuit interconnects 42.

Device 20a further includes one or more batteries 36 for supplying power to the device, and in some embodiments, and/or one or more exposed body electrodes 40 on an exterior surface of enclosure 32. One or more circuit interconnects 42 can provide the electrical coupling between the electronic components 34, one or more leads 28, electrode(s) 40, and batteries 36. Additional circuitry may be provided to facilitate recharging batteries 36.

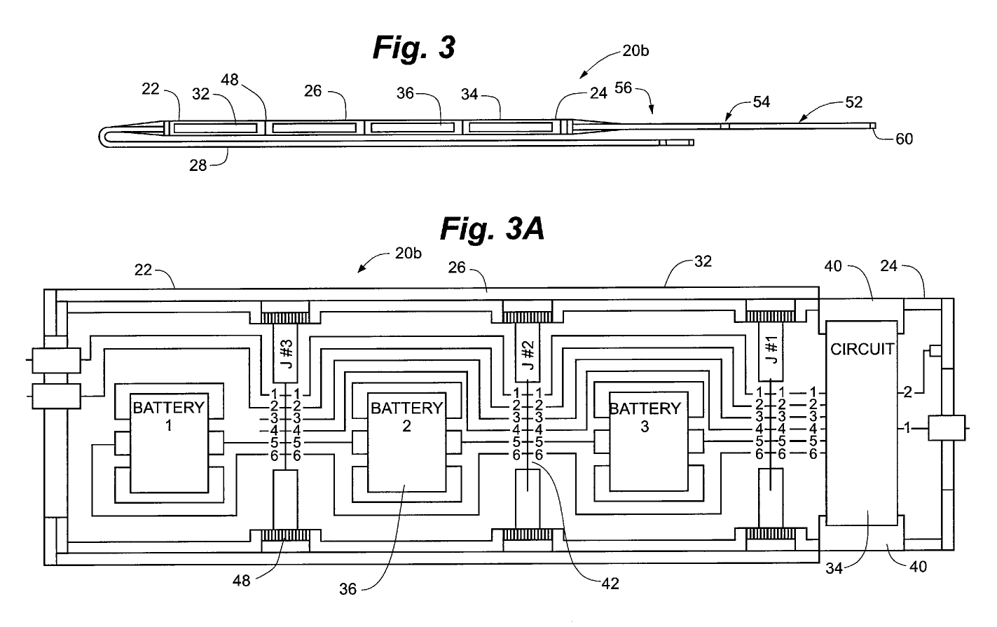

A second example of an arrangement of components for the intravascular implantable pacing device is identified by reference numeral 20b and shown in FIGS. 3-3A. As depicted in FIGS. 3-3A, the components of device 20b may be arranged in series with one another to give device 20b a streamlined profile. Because device 20b is intended for implantation within the patient's vasculature, some flexibility is desired so as to allow the elongate device to be easily passed through the vasculature. Flexibility may be added by segmenting device 20b, such as by forming one or more breaks in the enclosure, and by forming one or more hinge zones or bellows at each break which form dynamic flexible zones that can bend relative to the longitudinal axis of the device 20b in response to passage and/or positioning of device 20b though curved regions of the vasculature.

In device 20b, each segment may be separately enclosed by its own titanium (or similar) enclosure in the form of containers or compartments 32. The components within the containers 32 may be electrically connected by flexible circuit connects 42, for example. In one embodiment, the containers 32 are connected using a flexible material such as silicone rubber filler to form hinge zones. In another embodiment, flexible device 20 includes one or more rigid enclosures or containers 32 used to contain electronic components 34 to be implanted inside the vasculature of a patient and having the hinge zones formed of a bellows arrangement 48.

Containers 32 can be of any appropriate shape, cross-section, and length, but in this example are shown to have a cylindrical shape with a diameter of approximately 3-15 mm and a length of approximately 20 mm to 75 mm. Containers 32 can be used to house electromechanical parts or assemblies to form sophisticated implantable devices such as defibrillators, pacemakers, and drug delivery systems. Any appropriate number of these containers 32 can be combined using interconnecting bellows 48. Interconnecting mechanical bellows 48 can be used, to connect a number of rigid containers 32 in order to form a flexible device 20. For many devices, this will include a string of at least three containers 32. In one embodiment, the aspect ratio of the cross-sectional diameter to the longitudinal length of each container is less than at least 1.5:2 (e.g., 15 mm diameter to 20 mm length) and in another embodiment the aspect ratio is at least 1:4.

In one embodiment, the bellows 48 can be of any appropriate shape, but can preferably have a shape similar in cross-section to the cross-section of the container, in order to prevent the occurrence of edges or ridges that can give rise to problems such as the formation of blood clots in the vasculature. The bellows can be made of a biocompatible material similar to the containers. Any coatings used for electrically insulating the containers and/or making the containers more hemo-dynamically compatible also can be used with the bellows.

In addition to the ability of the bellows 48 to bend away from the central or long axis of device 20, the bellows 48 also allow for flexibility along the central axis of the device. The ability to flex along the central axis provides shock absorption in the long axis as well as 3-dimensional flexing. Shock absorption can help to protect device 20 and internal components during the implant process by minimizing the motion of the implanted device. Further, shock absorption can provide a 1:1 torque ratio for steering during the implant process. The shock absorption also can help during the life of device 20, as the natural movement of the body of a patient can induce some stress on the device 20.

For a more detailed explanation of the various embodiments of the bellows arrangements 48, reference is made to U.S. Published Patent Application Nos. 2006/0217779, filed Mar. 24, 2005, and 2007/0265673, filed Apr. 3, 2007, the disclosures of each of which are hereby incorporated by reference herein. Referring now to FIGS. 4-5A, another embodiment of the device, identified by reference numeral 20c, is depicted. Device 20c is similar to the embodiment depicted in FIGS. 3-3A, however device 20c includes multiple leads 28 on the proximal portion 22 of device 20c.

Referring now to FIGS. 6-6A, another embodiment of the device identified by reference numeral 20d is depicted. Device 20d is an intravascular implantable defibrillation device, having a lead 28 adapted to inserted into the right ventricle of a patient. Device 20d further includes one or more sensing electrodes, which may be located on the exterior of enclosure 32, similar to body electrodes 40. Device 20d also includes one or more defibrillation electrodes on the exterior of enclosure 32.

Referring again generally to device 20, the device is preferably able to communicate via wireless telemetry to an instrument outside of the patient's body. This is commonly referred to as device interrogation and/or programming and allows the physician to monitor the state and performance of the device. It also allows the physician to reconfigure the device in the case of programmable settings. The circuitry used for device interrogation and/or programming can be included in all of device 20 embodiments, with the device telemetry antenna either encapsulated within the device enclosure or as part of the tether 52 discussed in more detail below. The circuitry may include a circuit that will respond in the presence of a magnetic field, electric field, a near-field or a far-field, all which are features also known in the implantable device industry.

These communication techniques, either alone or in various combinations, are intended to allow device 20 to communicate the device's status to the physician. For example, the status information may include the state of the battery system, and whether or not a therapeutic energy delivery had occurred or not. The communication might also identify the parameters device 20 used, including stored electrograms, to allow reconstruction of the delivery episode by the instrument. The telemetry feature may also be used to program certain features governing function of device 20, such as the threshold heart rate in beats per minute which, when detected by the device, will cause the device to provide appropriate energy therapy.

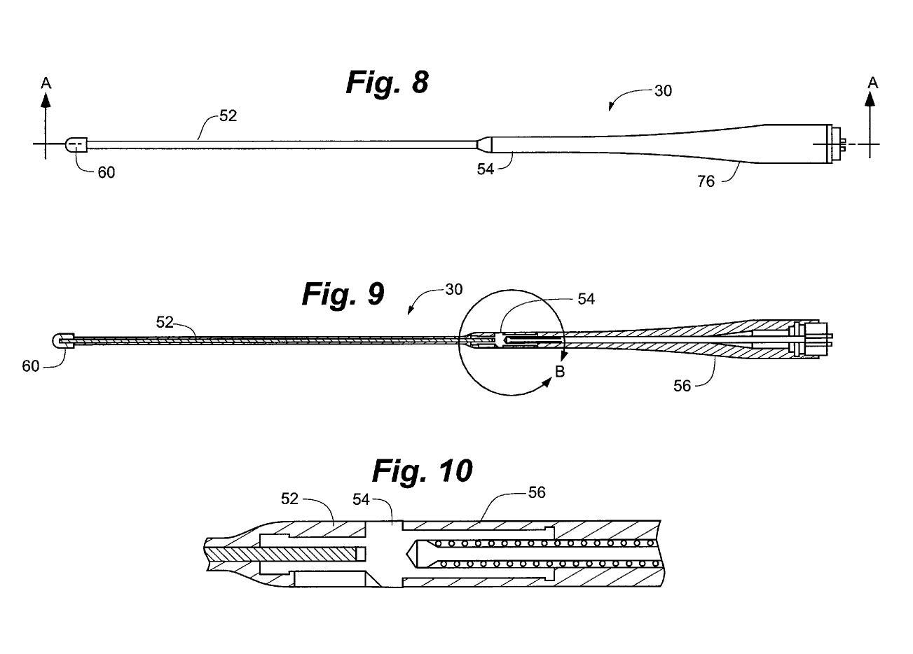

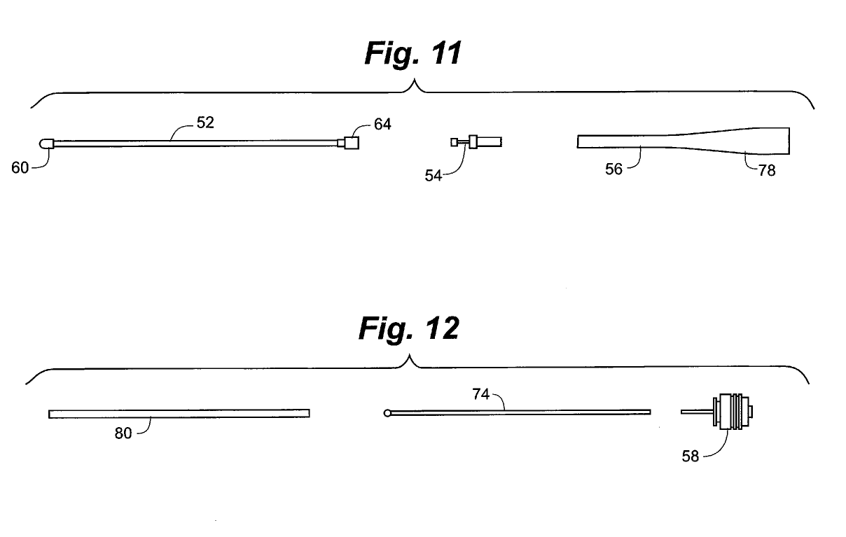

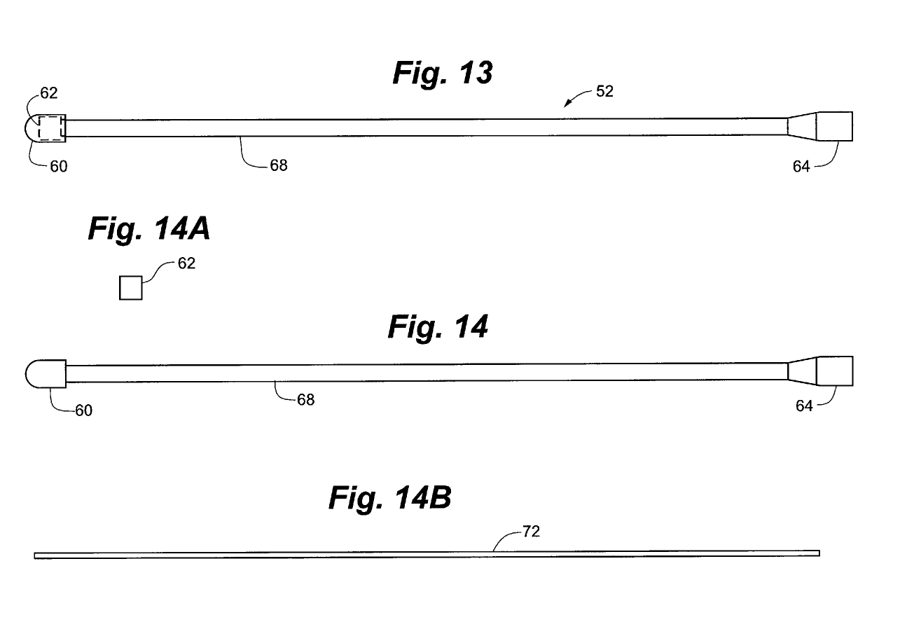

Referring now to FIGS. 8-14B, one embodiment of detachable tip assembly 30 and its various components are depicted. Detachable tip assembly 30 comprises a tether 52, a break-away joint 54, and an optional extension portion 56. Detachable tip assembly 30 is coupled to device 20 with connector 58. As used herein, detachable may comprise severable, releasable, or otherwise removable.

Tether 52 includes a tip portion 60, an optional locator ring 62, a connector portion 64, a lumen 66, and a housing 68. Tip portion 60 is configured to prevent tether 52 from being pulled out from an anchor 50 (discussed in further detail below). Tip portion 60 functions as a stop, interfering with the distal end of anchor 50 and preventing tether 52 from being pulled out from between anchor 50 and vessel wall 100. A locator ring 62 is optionally included on tip portion 60, and is configured to assist the implantation of device 20 by providing increased visibility of tether 52 in fluoroscopy visualization. Connector portion 64 is adapted to couple tether 52 to break-away joint 54 such that tether 52 does not separate from joint 54. Housing 68 is constructed of a suitable material such as ChronoFlex polyurethane, or ElastEon. Lumen 66 may be configured to receive a wire 72 for steering, or for structure and shape. Wire 72 may be composed of surgical-grade stainless steel, a molded polymer, or other materials apparent to one skilled in the art.

In one embodiment, break-away joint 54 is configured to be removably coupled to extension 56, and to be non-removably coupled to tether 52. In another embodiment, break-away joint 54 is configured to be removably coupled directly to device 20. Joint 54 may comprise a snap-fit, a threaded joint, or other similar configurations. One type of materials for joint 54 include surgical-grade stainless steel.

Extension portion 56 is optionally included in detachable tip assembly 30. Extension 56 comprises a housing 78, constructed of a suitable material such as ChronoFlex polyurethane, or ElastEon. Housing 78 is adapted to hold various components such as an antenna 74, or a lead 28 (not shown) for defibrillation, pacing, or sensing of cardiac electrical activity. The use of a lead in extension 56 may be especially useful in defibrillation to generate a shock vector across the heart. As discussed above, device 20 is preferably able to communicate via wireless telemetry to an instrument outside of the patient's body. In one embodiment, an antenna 74 may be provided within housing 78 to facilitate device interrogation and/or programming. An antenna isolation sleeve 80, constructed of dielectric material, may surround antenna wire 74. Antenna wire 74 may engage break-away joint 54, as depicted in FIG. 10. Extension housing 78 is adapted to non-removably couple joint 54, and may be coupled to joint 54 by adhesive, welding, soldering, or other suitable methods. Housing 78 may also be molded directly onto joint 54.

Extension portion 56 is particularly useful when it is desired to anchor device 20 within a patient's subclavian region, such as in the subclavian vein or cephalic vein. In one such embodiment, during implantation, device 20 is routed through the vena cava and up to the subclavian crush zone 111, which is defined as the region of the left (or right) subclavian vein that can be compressed between a patient's clavicle and first rib, due to upward movement of the patient's arm. Typically, when a large object (such as a device body or lead) is introduced intravascularly and is placed within subclavian crush zone 111, the object can become damaged, potentially leading to failure of the object or damage to the vessel. This problem is compounded if multiple leads or other intravascular devices are located within the crush zone, as there is a tendency for the leads and/or devices to abrade one another, resulting in an increased potential for failures of the leads and/or devices.

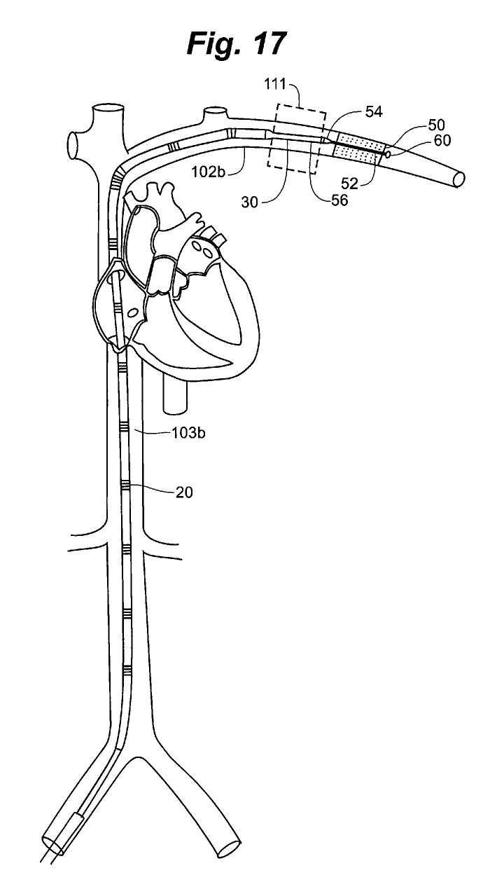

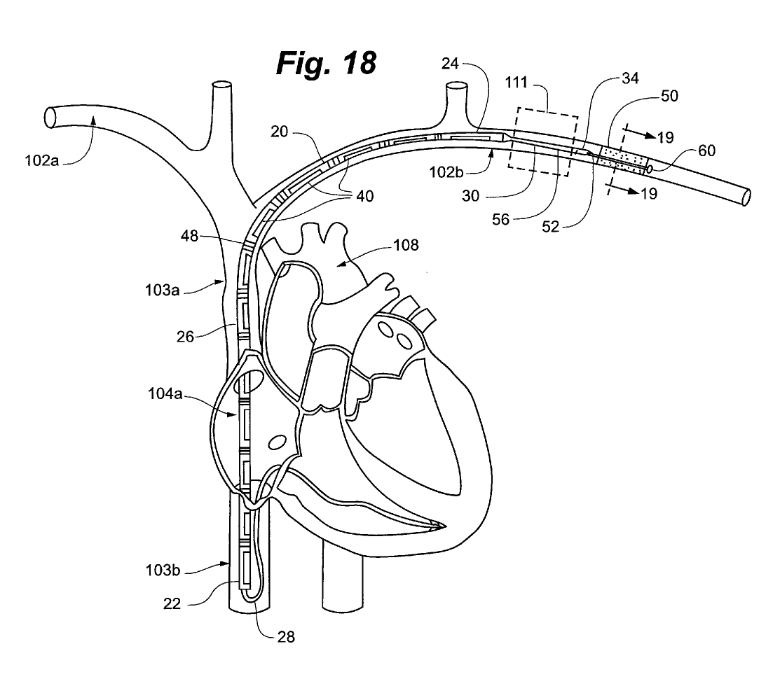

In such an embodiment, device 20 is positioned proximate subclavian crush zone 111 while extension portion 56 extends across and through the crush zone. Tether 52 is secured with an anchor 50 located beyond and peripheral of subclavian crush zone 111, for example as depicted in FIGS. 17 and 18. In another embodiment, anchor 50 may secure tether 52 within the patient's cephalic vein.

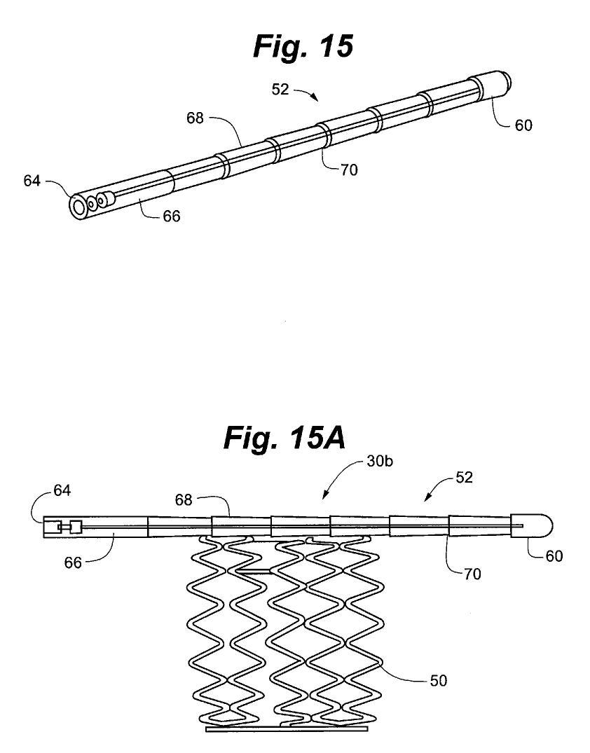

Referring now to FIGS. 15 and 15A, an embodiment of a detachable tip assembly 30b and its components is depicted, including a tether 52 having a tip portion 60, a plurality of barbs 70 disposed on the outer surface of a housing 68, a connector 64 and a lumen 66. Barbs 70 comprise structural features for anchor 50 to grip against, increasing the holding strength of tether 52 by anchor 50. Connecter 64 is configured to releasably couple tether 52 to device 20, such that the force required to separate device 20 from tether 52 is less than the force required to tear tether 52 from anchor 50. The distance between barbs 70 may be adjusted according to the design on anchor 50.

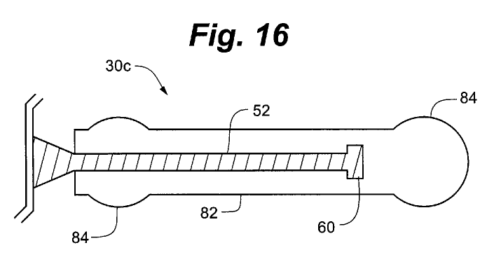

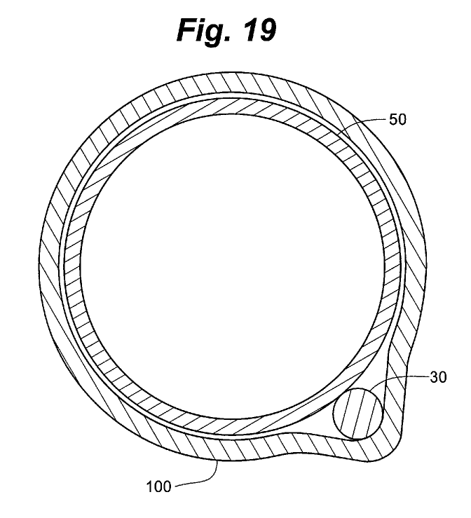

Referring now to FIG. 16, a further embodiment of a detachable tip assembly 30c and its various components is depicted. Detachable tip assembly 30c includes a tether portion 52 having a tip portion 60, and a sleeve 82. Tether portion 52 is non-removably coupled to device 20, and tether 52 is of a smaller diameter than device 20, as with the other embodiments discussed above. Sleeve 82 may be integrated with an anchor 50, or may be separable from the anchor. Sleeve 82 is preferably sandwiched between an anchor 50 and a vessel wall 100, similar to the embodiment depicted in FIG. 19.

Sleeve 82 may be constructed of a molded polymer, for example, or other biocompatible materials apparent to one skilled in the art. Sleeve 84 includes at least one structural retention feature 84. As depicted in FIG. 16, an anchor 50 is configured to engage sleeve 82 between the two structural retention features 84, insuring sleeve 82 is held firmly in place. Sleeve 82 is configured to releasably receive tether 52 therein. Tether 52 is detachable from sleeve 82, although sleeve 82 features an inner profile configured to secure tether 52 during normal use by a patient. If explantation of device 20 is required, pulling device 20 away from sleeve 82 with sufficient force will result in tether 52 disengaging from sleeve 82. The interface between tip portion 60 and sleeve 82 is such that under normal conditions it is not possible for tether 52 to “fall out” of sleeve 82.

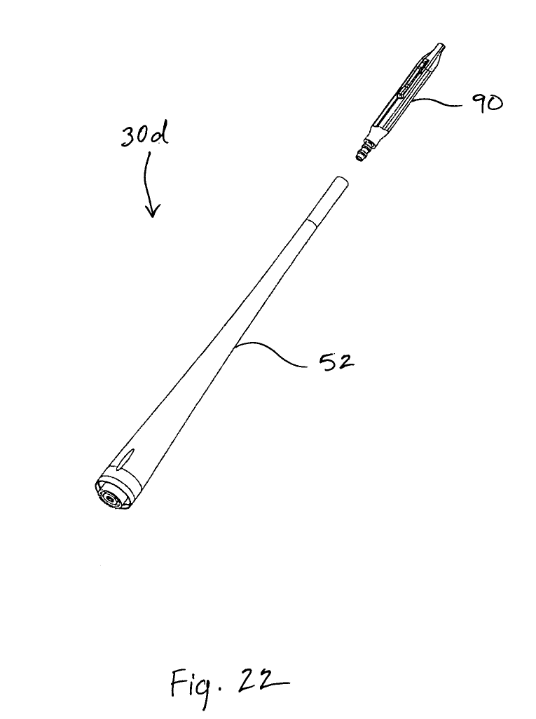

Referring now to FIG. 22, a detachable tip assembly 30d is depicted, comprising a tether 52 and a cleat 90. In one embodiment, tether 52 is detachable from the device body. In another embodiment, cleat 90 may be detachable from tether 52. Cleat 90 includes one or more clips adapted for mechanically securing cleat 90 to an anchor 50.

Referring now to anchor 50, it is configured to releasably retain device 20 within a patient's vasculature such as in a subclavian vein, cephalic vein, jugular vein, or in the superior vena cava. In one embodiment, anchor 50 comprises a conventional intravascular stent. Tether 52 may be secured by being “sandwiched” between a vessel wall 100 and anchor 50, as best depicted in FIG. 19. In one embodiment, anchor 50 is separable from tether portion 52, although anchor 50 may also be integrated with tether portion 52.

In one embodiment, anchor 50 may include features that give some structural stability to cause the anchor to radially support device 20 against a vessel wall 100. For example, a mesh or other framework formed of shape memory (e.g. nickel titanium alloy, nitinol or shape memory polymer) elements or stainless steel wires may be used to form anchor 50. In another embodiment, the anchor 50 is provided with a smooth polymeric barrier that is both anti-proliferative and anti-thrombogenic and that thereby prevents endothelial growth and thrombus formation on the anchor. Examples of materials for the polymeric barrier include, but are not limited to ePTFE, or other fluoropolymers, silicone, non-woven nylon, or biomimetic materials. The polymeric barrier on anchor 50 is preferably formed by layers of barrier material on the interior and exterior surfaces of the framework, although it will be appreciated that the framework and barrier may be combined in a variety of ways to prevent thrombus formation and endothelialization on the anchor walls.

As one alternative (or in addition to the polymeric barrier), the anchor material could include surfaces for eluting non-coagulative, anti-platelet (e.g. IIBIIIA glycoprotein receptor blockers), anti-proliferative, and/or anti-inflammatory substances. Additional information pertaining to the construction, materials and operation of anchors suitable for use with the present invention are described in U.S. Pat. No. 7,082,336 and U.S. Published Patent Application No. 2004/0249431, the disclosures of each of which are hereby incorporated by reference herein.

Detachable tip assembly 30 is preferably of a smaller diameter than device 20. Preferably, extension portion 56 is a smaller diameter than device 20, and tapers down to a still further diameter to match the diameters of joint 54 and tether 52, as best depicted in FIGS. 8-9. Minimizing the diameter of tether 52 may be desirable so as to reduce bulging and/or irritation of the vessel wall 100. In embodiments where device 20 comprises a defibrillator, tether 52 is likely shorter in length than the defibrillator. In embodiments where device 20 comprises a pacemaker, tether 52 may be shorter than, longer than, or similar in length to, the pacemaker.

Tip assembly 30 may be somewhat flexible to allow bending during implantation, yet is also somewhat rigid. In one embodiment, tether 52 includes a wire 72 that provides rigidity. Retention tip 60 on tether 52 is configured to prevent tether 52 from being pulled out from anchor 50. Tip 60 functions as a stop, interfering with the distal end of anchor 50 and preventing tether 52 from being pulled out from between anchor 50 and vessel wall 100.

Referring now to the implantation of device 20, specific details of various implantation embodiments are discussed in U.S. Provisional Application titled “Implantation Methods, Systems and Tools for Intravascular Implantable Devices”, filed Dec. 3, 2007, the disclosure of which has been incorporated by reference herein.

In one general embodiment, device 20 is implanted by making an incision in the patient's femoral vein, and inserting an introducer sheath through the incision into the vein. The introducer sheath keeps the incision open during the procedure, and includes a seal adapted to prevent blood from exiting the body while allowing the insertion of various tools and devices into the body. Device 20 may be introduced in a number of ways. In one embodiment, the device 20 may be introduced by an over-the-wire technique. The distal end or distal portion of device 20 is provided with a passageway configured to receive a guidewire, and the device is slid onto the guide wire, then the distal end of device 20 is introduced through the seal. Device 20 is guided through the vasculature of the patient, into the inferior vena cava, then the superior vena cava, and into the subclavian vein or other vessel superior to the heart. A device delivery catheter may be used to facilitate introducing the device.

Next, the anchor 50 is introduced. Anchor 50 may be inserted through the seal in the femoral incision used to implant device 20. In another embodiment, anchor 50 is inserted from another incision such as through an incision closer to the location of tether portion 52. In one embodiment, anchor 50 may be introduced after device 20 has been positioned at the desired location within the vessel. In another embodiment, anchor 50 may be introduced prior to device 20 being introduced.

Referring to an embodiment wherein anchor 50 is introduced via the femoral incision, the anchor may be delivered over the guide wire, such as with an anchor delivery catheter. Anchor 50, compressed to a streamlined position, is passed through the vasculature and approaches the distal portion of the device where the anchor will interfere with where the wire enters the tip of the device. The guide wire must be removed from the device and guided around the tip of the device to provide a path for the anchor. The anchor is then guided around the device and past the distal-most portion of the device tip. Anchor 50 may be self-expanding and/or it may be expanded using an inflation tool such as a balloon passed into the anchor's central lumen and subsequently inflated. When anchor 50 is expanded, its radial forces engage tether portion 52 and secure tether portion 52 against vessel wall 100, as depicted in FIG. 19. Depending on the characteristics of anchor 50, the expansion force of the anchor against tether portion 52 may cause the vessel wall 100 to bulge outwardly. Alternatively, the anchor 50 may deform around the shape of tether portion 52, leaving vessel 100 at its normal shape. In another embodiment, both anchor 50 and vessel 100 deform to accommodate tether portion 52. It is desirable to minimize the diameter of tether portion 52, to minimize deformation of anchor 50 and/or vessel 100.

Referring now to FIGS. 17-21, various example installations of a device 20 having a detachable tip assembly 30 are depicted. Generally speaking, suitable locations for anchoring device 20 can include, but are not limited to, inferior vena cava 103b, superior vena cava 103a, either subclavian vein, either innominate/brachiocephalic vein, either jugular vein, and either cephalic vein. Detachable tip assembly 30 may be located on distal portion 24 of device 20, or on proximal portion 22. Additional information regarding anchoring, including tools, techniques and suitable locations superior to the heart can be found in U.S. Provisional Application No. 60/868,437, filed Dec. 4, 2006, the disclosure of which has been incorporated by reference.

Referring now to FIG. 17, one embodiment of a device 20 is depicted having a tip assembly 30 located at distal portion 22. Tip assembly 30 includes an extension portion 56 between device body 20 and detachable tether portion 52. An anchor 50 is secured within left subclavian vein 102b. It will be appreciated that although not pictured, tether portion 52 could be made longer so as to anchor tether 52 within the patient's cephalic vein, while maintaining the location of device 20 proximate subclavian crush zone 111.

Referring now to FIG. 18, one embodiment of a device 20 is depicted having multiple exposed body electrode regions 40. Exposed body electrode regions 40 may include defibrillation electrodes and/or sensing electrodes, for example. The activation of body electrodes 40 may be adjusted and controlled by the various circuitry within device 20 as needed.

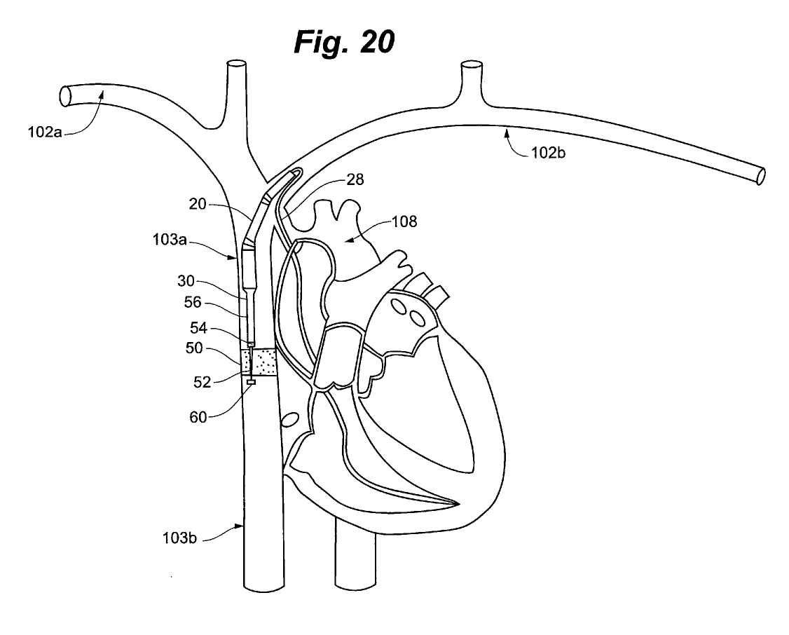

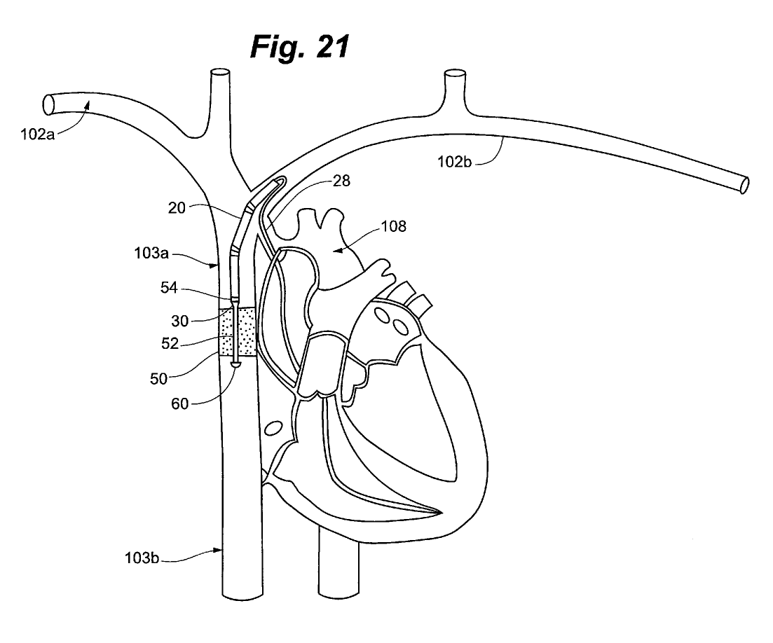

Referring now to FIGS. 20 and 21, two similar embodiments of device 20 are depicted. In FIG. 20, device 20 includes a detachable tip assembly 30 having an extension portion 56, while the device 20 in FIG. 21 includes a detachable tip assembly that lacks an extension portion 56. The embodiments of FIGS. 20 and 21 are anchored within superior vena cava 103a.

There are a number of options for separating the device from some or all of the tip assembly. In one embodiment, the device may be cut away from the tip assembly during an intravascular procedure. A catheter containing a cutting mechanism is advanced through the vasculature to the desired location, and some part of the tip assembly is cut, releasing the device. In another embodiment, the connection between the device and the tip assembly is designed such that if a sufficient pull force is exerted on the device, the device will separate from the tip assembly. A suitable force required to separate the device and tip assembly should be large enough to not allow the device to accidentally separate from the tip assembly, yet low enough to disengage the connection rather than damage the anchor's fixation in the vessel.

In one embodiment, the connection between the device and the tip assembly may be threaded, with one component serving as a screw and the other component the threaded bore. Rotation of the device, such as by a grasper on the distal end, may separate the device and tip assembly, allowing removal of the device.

In another embodiment, one or more magnets are provided in the device and/or tip assembly to connect the device and tip assembly. The one or more magnets may be rotatable, such that causing the magnet to rotate changes a magnetic attractive force between two adjacent components to a magnetically repulsive force, thereby detaching the tip assembly from the device. In another embodiment, a magnet may be provided in conjunction with a mechanical fastener, such as a screw, so that by rotating the magnet causes the screw to rotate, separating the device and tip assembly.

In another embodiment, a motor or other motive force actuator is provided, such as in the tip assembly. The motor or motive force actuator may be coupled to a screw, and powering the motor results in the separation of the device and tip assembly.

Other arrangements of detach mechanisms will be appreciated by one skilled in the art, such as quarter- or half-turn connections between the device and tip assembly, or solenoid actuators, or piezoelectric actuators. Alternatively, pronged or spring-loaded clips may be engaged or disengaged to effect a release of the detachable tip.

It should be pointed out that many of the device configurations, components, retention devices and methods, implantation methods and other features are equally suitable for use with other forms of intravascular implants. Such implants might include, for example, implantable neurostimulators, artificial pancreas implants, diagnostic implants with sensors that gather data such as properties of the patient's blood (e.g. blood glucose level) and/or devices that deliver drugs or other therapies into the blood from within a blood vessel.

Various embodiments of systems, devices and methods have been described herein. These embodiments are given only by way of example and are not intended to limit the scope of the present invention. It should be appreciated, moreover, that the various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the invention.

For purposes of interpreting the claims for the present invention, it is expressly intended that the provisions of Section 112, sixth paragraph of 35 U.S.C. are not to be invoked unless the specific terms “means for” or “step for” are recited in a claim.

1. An intravascular device, comprising:
an elongate device body with a first end and a second end that is adapted for chronic implantation within the vasculature of a patient;
a detachable tether that extends beyond the first end of the elongate device body; and
means for anchoring the detachable tether within the vasculature of the patient without mechanically securing the elongate device body within the vasculature of the patient,
such that the elongate device body is extractable from the vasculature of the patient when the detachable tether is detached from the device body.

2. The intravascular device of claim 1 wherein the intravascular device is adapted to be inserted into the vasculature of the patient through a femoral vein and the first end of the device body is a distal end of the device body and the second end of the device body is a proximal end of the device body.

3. The intravascular device of claim 1 wherein the detachable tether has an average cross-sectional diameter that is smaller than an average cross-sectional diameter of the elongate device body.

4. The intravascular device of claim 1 wherein the detachable tether comprises:
a tip assembly including a break-away joint and a tether portion; and
an extension portion that forms an integral part of the first end of the elongate device body,
such that the break-away joint is longitudinally disposed between the extension portion and the tether portion.

5. The intravascular device of claim 4 wherein the break-away joint comprises one of a mechanically coupled joint, a motive power coupled joint, or a combination thereof.

6. The intravascular device of claim 4 wherein the extension portion includes at least one of an antenna adapted to transmit and receive data, an electrode adapted to deliver electrical pulses, an electrode adapted to sense cardiac electrical activity, or a combination thereof.

7. The intravascular device of claim 4 wherein the extension portion has a tapered cross-sectional diameter that generally tapers between a cross-sectional diameter of the device body and cross-sectional diameter of the tether portion.

8. The intravascular device of claim 4 wherein the extension portion includes a lumen having an access port defined in a side wall of the extension portion and is adapted to receive a guide member within the lumen.

9. The intravascular device of claim 8 wherein the tether includes a lumen having an exit port defined in the tether and the lumen of the tether portion is coupled to the lumen of the extension portion such a guide wire as the guide member threaded through the lumens enables over-the-wire implantation of the intravascular device.

10. The intravascular device of claim 2 wherein the tether extends beyond the distal end of the elongate device body and the tether is adapted to be intervascularily positioned in a target vessel located beyond a subclavian crush zone of the patient in a direction away from the heart without the elongate device body being advanced into the subclavian crush zone.

11. The intravascular device of claim 1 wherein the tether includes structure that interfaces with structure on means for anchoring to mechanically engage the tether with the anchor.

12. The intravascular device of claim 11 wherein the tether includes a cleat having a pair of laterally opposed structures on the tether, either of which are adapted to interfaces with structure on the means for anchoring.

13. The intravascular device of claim 12 wherein the cleat includes a pair of laterally opposed clip structures and a corresponding pair of laterally opposed fin structures orthogonally offset from an orientation of the clip structures, wherein the means for anchoring is a radially expandable stent having a plurality of struts that define vertices at intersections thereof, such that when the device body is rotated the fin structures orient one of the clip structures to interface with one of the vertices of the plurality of struts whereby the one of the clip structures engages with the one of the vertices of the plurality of struts when the device body is pulled back relative to the anchor.

14. A method of providing an intravascular device and instructions for implanting the intravascular device, comprising:
providing an intravascular device having an elongate device body with a first end and a second end that is adapted for chronic implantation within the vasculature of a patient and a detachable tether that extends beyond the first end of the elongate device body;
providing instructions for chronically implanting the intravascular device substantially wholly within the vasculature of a patient, the instructions including:
introducing the intravascular device into the vasculature of the patient;
advancing the intravascular device until the tether is positioned within a target vessel of the patient;
anchoring the tether within the target vessel without mechanically securing the elongate device body within the vasculature of the patient; and
providing instructions for extracting the intravascular device from the vasculature of a patient, the instructions including:
detaching the detachable tether from the device body; and
removing the intravascular device from the vasculature of the patient while leaving the tether within the vasculature of the patient.
